(19) United States
(12) Patent Application Publication
(10) Pub. No.: US 2010/0174559 A1
(43) Pub. Date: Jul. 8, 2010

(54) METHOD AND SYSTEM FOR PROVIDING NEURON CONDITIONING

(76) Inventor: WILLIAM J. McFAUL, Jackson, NJ (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 12/651,763

(22) Filed: Jan. 4, 2010

Related U.S. Application Data
(63) Continuation-in-part of application No. 11/778,966, filed on Jul. 17, 2007.
(60) Provisional application No. 60/832,633, filed on Jul. 21, 2006.

(51) Int. Cl. G06Q 50/00; A61B 5/055

(52) U.S. Cl. 705/3; 600/410

(57) ABSTRACT

A method and system for forming an integrated group. The method comprises taking and comparing neuro-hormone ratios of individuals and comparing the ratios to predetermined ratio. The method may also comprise comparing an fMRI scan of an individual to a predetermined fMRI scan. By correcting differences from the predetermined values integrated groups may be formed.

This application is a continuation-in-part of U.S. application Ser. No. 11/778,966 filed Jul. 17, 2007, which in turn is the non-provisional application of U.S. Provisional Application No. 60/832,633 filed on Jul. 21, 2006, the entireties of which are incorporated herein by reference.

### BACKGROUND OF THE INVENTION

1. Field of Invention

The invention relates to a method of conditioning neurons. In particular, the invention relates to a method of conditioning a plurality of neurons so as to have a similar pattern over an extended duration of time.

2. Description of Related Technology

Whenever there is an attempt to bring two or more organizations together to foster collaboration within a company, it is inevitable that an array of issues will arise due to conflicting outlooks by the groups. Organizations will differ in a range of different ways that reflect their underlying cultural differences. Integrating the organizations so that behavior and thinking skills coalesce has proven to be a daunting task.

Typical solutions have focused on compelling an organization to adopt the behavior and thinking skills of the dominant group. The organization being dominated tends to reject or conform in a matter that renders the adopted behaviors ineffective or inappropriately utilized.

Furthermore, today's healthcare, scientific research, and financial organizations have business, clinical objectives and geo-political environments that demand optimally efficient planning and decision support processes for all activities. Unfortunately, due to time constraints and the often conflicting and confusing cross-cultural communication, terminology differences, and management theories, organizations rarely have the opportunity to carefully analyze the array of options available to meet their specific needs and adapt them appropriately.

Therefore there is a need in the field to provide a method and system for of conditioning a plurality of neurons so as to have a similar pattern over an extended duration of time.

### SUMMARY OF THE INVENTION

Another aspect of the present invention can be a collective educational system comprising at least one organization; a plurality of members selected to form an internal consulting group; an instructor; a network connecting said plurality of members enabling communication and collaborative learning.

An aspect of the present invention may be a method for testing for an integrated group comprising the steps of measuring a first neuro-hormonal level of an first individual; determining a first ratio of neuro-hormones based upon the first neuro-hormonal level; compiling a plurality of ratios, wherein the first ratio of neuro-hormones is one of the plurality of ratios; comparing the first ratio with a predetermined ratio; an correcting the first ratio of the first individual.

Another aspect of the present invention may be a method for testing for an integrated group comprising the steps of: applying an fMRI reading to a first individual in order to obtain a first scan; evaluating the first fMRI scan; compiling the results of a plurality of fMRI scans; comparing the first fMRI scan with a predetermined fMRI scan; and correcting the first individual with respect to the predetermined fMRI scan.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which fowl a further part hereof, and to the accompanying descriptive matter, in which there is illustrated and described a preferred embodiment of the invention.

### BRIEF DESCRIPTION OF THE DRAWINGS

FIG. 1 is a diagram of the brain.

FIG. 2(a) shows a neural pattern of an individual's brain.

FIG. 2(b) shows the neural pattern of the individual's brain in FIG. 2(a) after the collaboration process.

FIG. 3(a) shows a neural pattern of an individual's brain.

FIG. 3(b) shows the neural pattern of the individual's brain in FIG. 2(a) after the collaboration process.

FIG. 4 shows a sample test used for an operations director.

FIG. 5(a) shows the individuals that form the integrated group.

FIG. 5(a) shows the integrated group.

FIG. 6 shows the flow chart of the method for evaluating skills.

FIG. 7 shows the flow chart of the method for evaluating characteristics.

FIG. 8 shows the flow chart of the method for evaluating knowledge.

FIG. 9 shows the flow chart of the method for evaluating mental functioning activity.

FIG. 10 shows the flow chart of the method for measuring the neuro-hormonal activity of an individual.

FIG. 11 shows method for evaluating the fMRI.

FIG. 12 shows the method of creating the integrated group.

FIG. 13 shows an example of the information, skills, knowledge topics and subjects that may be pursued by an organization.

FIG. 14 is a flow chart illustrating the steps used by an integrated group.

FIG. 15 is a skills evaluation.

FIG. 16 is another example of the evaluation of skills.

FIG. 17 shows an evaluation for characteristics.

FIG. 18 shows a sample examination for neurohormone knowledge.

FIG. 19 shows an evaluation of expense reduction internal consulting groups.

FIG. 20 shows an alternative examination for MFA.

### DETAILED DESCRIPTION OF THE INVENTION

The present invention is directed to affecting the neuro-hormonal levels of individuals to reorganize the neural wiring. The neural wiring of an individual is reshaped so to substantially similar to another individual. Ideally the neural wiring adjustment affects only select areas of the brain, preferably those areas that control the behavior and thinking patterns desired to be affected. The method employs finding similar behaviors and thinking patterns and creating an integrated set of behaviors and thinking patterns that are then expressed in the neural wiring of the individuals partaking in the method.

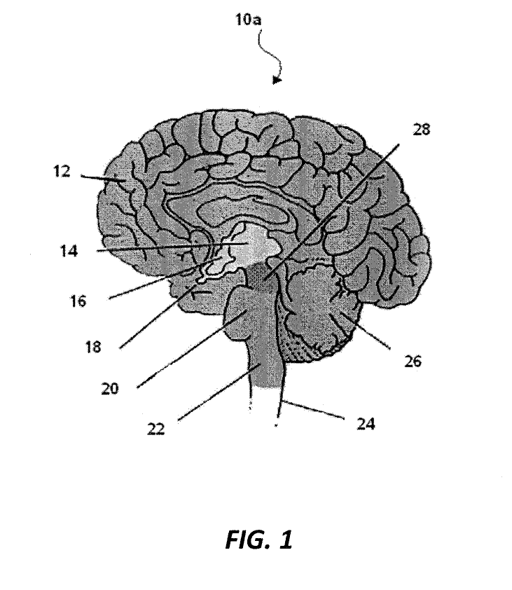

FIG. 1 shows a diagram of an individual's brain 10a Areas of the brain are pointed to such as cerebral hemisphere 12, thalamus 14, hypothalamus 16, amygdala 18, the pons 20, the medulla 22, the midbrain 28, the cerebellum 26 and the spinal cord 24. These areas control the behavior and thinking patterns of an individual. The neuro-hormonal activity that is affected and controlled primarily affects the cerebral hemispheres.

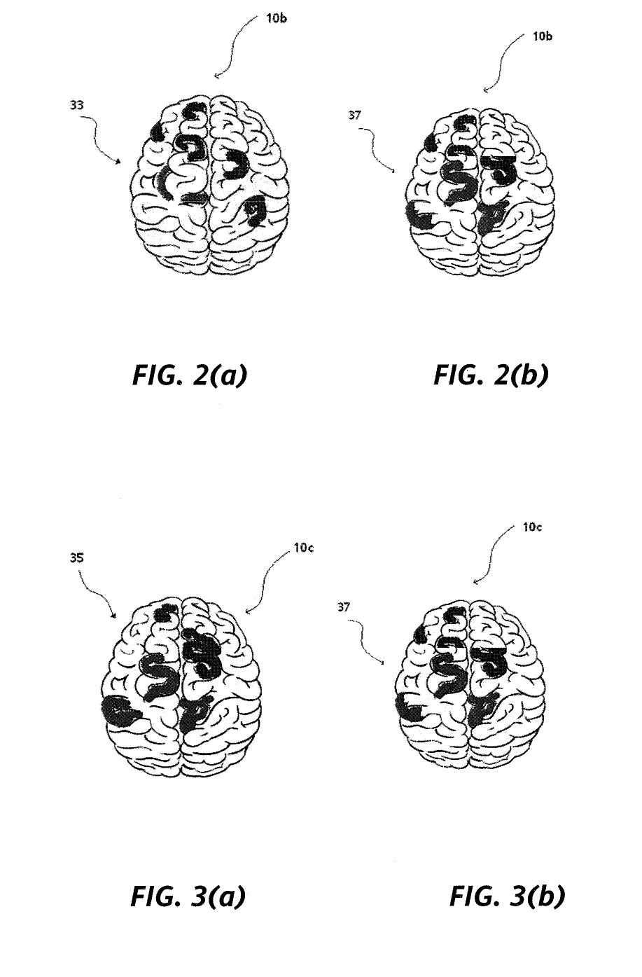

FIGS. 2(a)-3(b) illustrate the neural activity of two individual's brains, 10(b) and 10(c). The activity in brain 10(b) is shown by neural pattern 33, which is taken from an fMRI scan. The activity in brain 10(c) is shown by the neural pattern 35, which is taken from an fMRI scan. The resultant neural pattern 37, which is taken from an fMRI scan, is formed after the implementation of the integration process and is substantially similar in both brain 10(b) and brain 10(c). This similarity of neural patterns is the result of the determination of common features between individuals and the blending of the features together in order to form a common neural pattern 37 in each of the individuals within a particular group. This resultant group can be utilized in order to facilitate problem solving and fostering innovation in a variety of areas.

By using a variety of tools an organization can effectively form an integrated group that can restructure the organization in order to establish homogenous patterns of behavior and thinking skills. These tools include but are not limited to the use of comprehensive state-of-the art educational topics germane to techniques to enhance listening skills, non-verbal communication, win-win negotiating, group dynamics, the psychology of change, stress management as well as enabling the individuals to select the specific attributes from each of the specific educational topics that they, through consensus, decide would enable their internal consulting group to function optimally in an integrated manner. The creation of a common habit of thought and behavior by individuals will assist in the formation of neural wiring patterns.

Tests designed to evaluate the characteristics; the skills and the knowledge of individuals are used in order to establish the homogenous patterns of behavior and thinking skills that are found in common. Additionally, mental functioning activity (MFA) evaluations, and physically based tests, such as fMRIs of the brain and neuro-hormonal evaluation, may be used.

MFA is a term used to illustrate a spectrum of possible mental and behavioral characteristics that are correlated with their actual or categorized brain activity. MFA is evaluated on a scale that indicates possible behavioral and physical traits that stem from the underlying metabolic rate of the brain. For example some characteristic affected by MFA may be focus, over-focus such as obsession, awareness and disinterest, temperament such as rage, anger, aversion, and disenfranchisement; concern, such as fright, fear, sadness and despair; happiness, such as resentment, bitterness, joy, envy and avoidance. Physical traits affected by MFA may be overly-active, active, lethargic and sedentary. Obtaining the MFA of individual provides a gauge that helps ascertain a person's ability to dedicate greater or lesser focus on tasks and their ability to handle tasks. Furthermore, the characteristics and physical traits discussed above may have specific MFA levels attributed to them.

In general, MFA is more intimately related to the physical functioning of the brain and may be further visualized using magnetic resonance imaging or other physical diagnostic tools. However, in the event that the usage of physical imaging cannot be used, it is possible to use specially formatted questionnaires in order to gauge the level of a person's MFA.

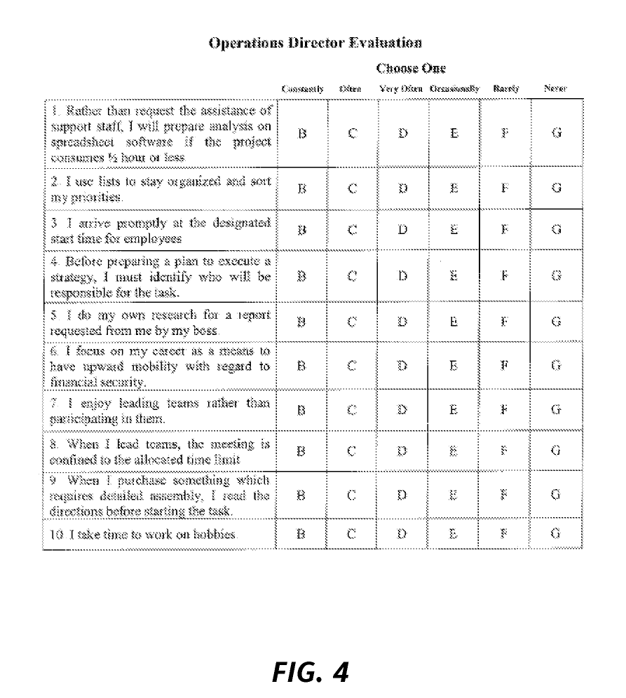

At example questionnaire that may assist in determining MFA, is found in FIG. 4. The questionnaire is a sample test that may be used to evaluate the MFA for an operation director. This test is a customized example of a general test that may be used in a generic business setting but can be modified to reflect individuals participating in a wide variety of scientific or medical research domains. The questions may be selected from a “master” worksheet, or a database of acceptable questions that are designed to appropriate gauging of MFA. Alternatively, the questions may be developed specifically for the sought after position.

Upon being tested a person may be given a categorization number such as 10-11-12-1-2-3, as a result of completing the test. These numbers are based upon positions of a clock. For example, under the topic of “leadership,” an individual selecting “collaboration” as their most likely assumed “role” as it relates to that topic would fall into the “12” category. This is based upon a pre-determined assignment of “position” for various characteristics. The sample test questions found in FIG. 4 have a customized sampling of topics chosen for relatedness to the requirements of the position. It should be understood that using a clock as the basis for the scale is a useful way to scale the resultant numbers. Other scales may be used that are not as intuitive in grasping the relatedness of various scores, but that are just as informative in the ultimate number obtained.

The determination of MFA may also be accomplished via the usage of an fMRI machine. By categorizing the images of individuals with specific behavioral and physical traits certain scans will correlate to different numbers.

Another test may alternatively, or in addition to the MFA test, be a test directed towards evaluating the characteristics, skills, and knowledge sets of individuals in order to provide an initial evaluation that considers multiple factors of people. One of the factors used is the characteristic set (C Set) of an individual. The characteristics of an individual are those traits that govern how an individual will act in certain environments, indeed it may focus upon an individual's MFA as discussed above.

An individual's skill set (S Set) is the second additional factor used in this method and system. An individual's S set are those traits that can be acquired through schooling or acquired through on the job training.

An individual's knowledge is the third factor used in this approach. An individual's knowledge set (K Set) is an element verifiable through proven accomplishments. Both skills and knowledge are essential considerations in evaluating a person's qualifications. However since skills and knowledge can be developed, i.e. acquired or learned, they serve to compliment a person's characteristics. The determination of a person's CSK set may assist in formation of an integrated group 50.

The integrated group 50 shares the common behaviors and thinking skills that are extracted from the component individuals that form the group. During the process of enabling the group to create its own operating mechanisms and protocols, the participants will have the time to recognize the skills and capabilities of their counterparts. As issues emerge that require particular skills, the group as a whole will be able to identify the individual or individuals most capable to address the needs of the group as a whole. This shared behavior and thinking skills are reflected in the rewiring of the neural pathways in the individuals found within the group. This is reflected in the patterns that can be measured via the usage of fMRIs and other neural imaging equipment.

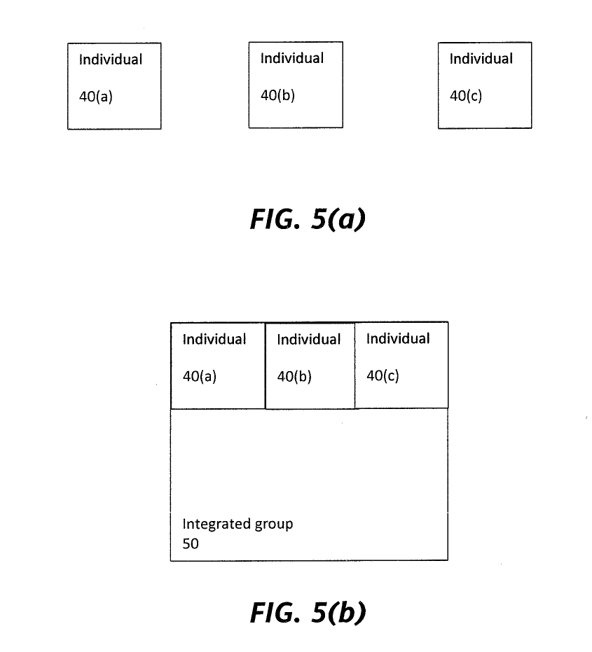

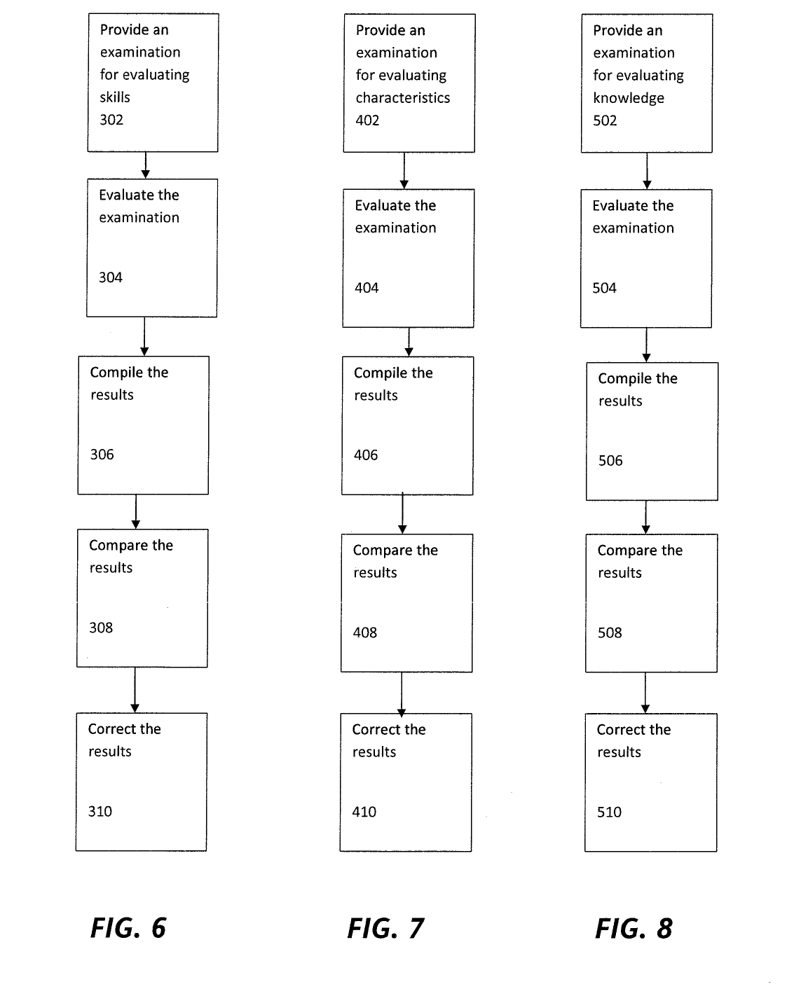

FIG. 5(a) illustrates the initial arrangement of individuals. Each of these individuals 40(a)-40(c) may come from different areas, backgrounds and organizations. These individuals may be subjected to testing in order to determine their base scores. FIGS. 6-11 show a number of testing processes. Each test applied will have a normative process that results in a specific score or spectrum that is then utilized to compare the individuals to each other. Tests may evaluate skills, characteristics and knowledge. The tests may also evaluate those physical characteristics and features of an individual that may also be indicative of these features. Tests used to determine mental functioning activity, tests used to determine specific neuro-hormonal levels and ratios and tests used to determine specific neural firing patterns that may be expressed via fMRI images of the brain may also be used.

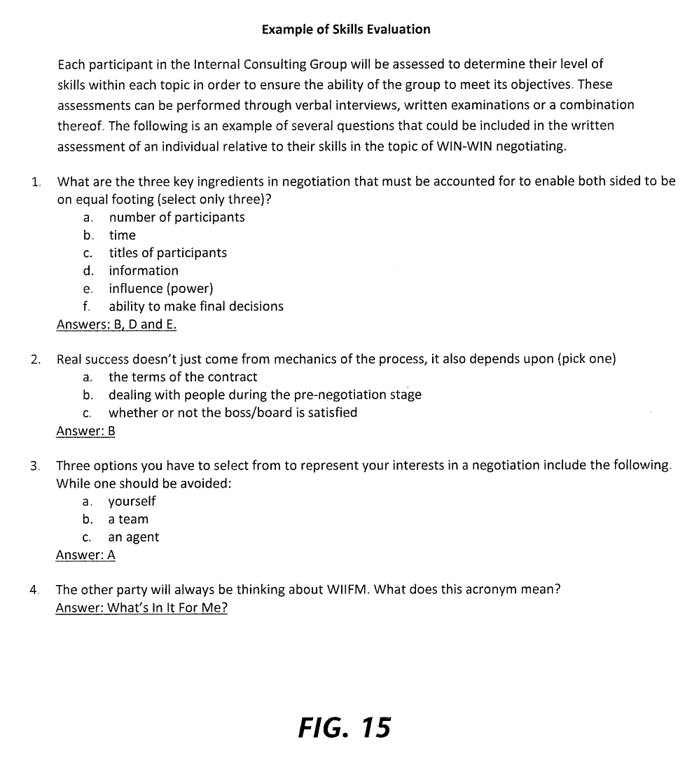

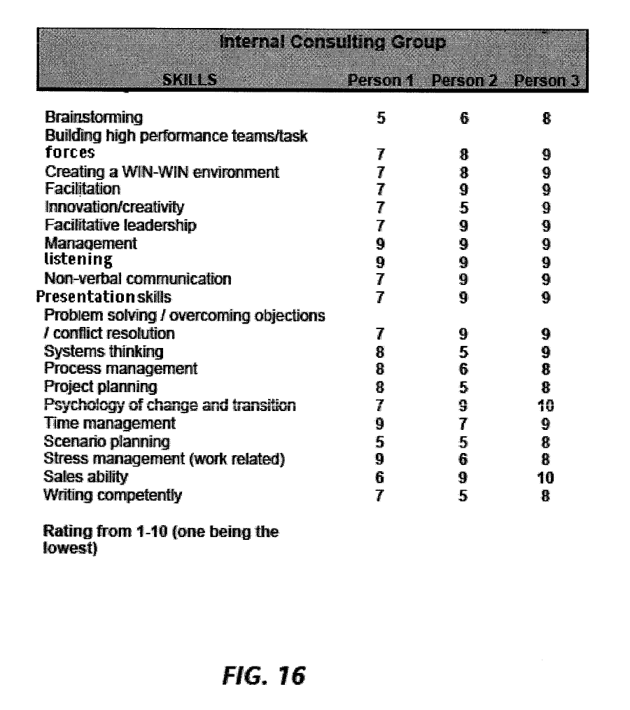

FIG. 6 shows the method for evaluating the skills of an individual. The method discussed in FIG. 6 is exemplary only and uses the diagrams shown in FIG. 5(a) and FIG. 5(b) in order to discuss the results. In step 302 an examination for evaluating skills is provided to an individual. FIG. 15 shows an example of skills evaluation. The skill examination determines the level of skill obtained by an individual 40(a) in areas such as group discussion, and the examples shown in FIG. 16. In step 304 the results of the skill test are evaluated. The result of this evaluation usually results in a number or some form of quantitative result. The quantitative result is indicative of the neural wiring that an individual currently has. In step 306 the results of the evaluation performed in step 304 are compiled with the results of the other individuals 40(b) and 40(c). In step 308 the results are compared with the others. A target number may be used in order to perform the comparison. For example the skill test may require a score of 50 out of a possible 100. Two of the individuals may only score a 40 and a 45, while the third may score a 55. This would indicate that the skill level of two of the individual's would need to be brought up to parity before formation of the integrated group 50. In step 310 the results are corrected. This may occur via providing specific training for that skill.

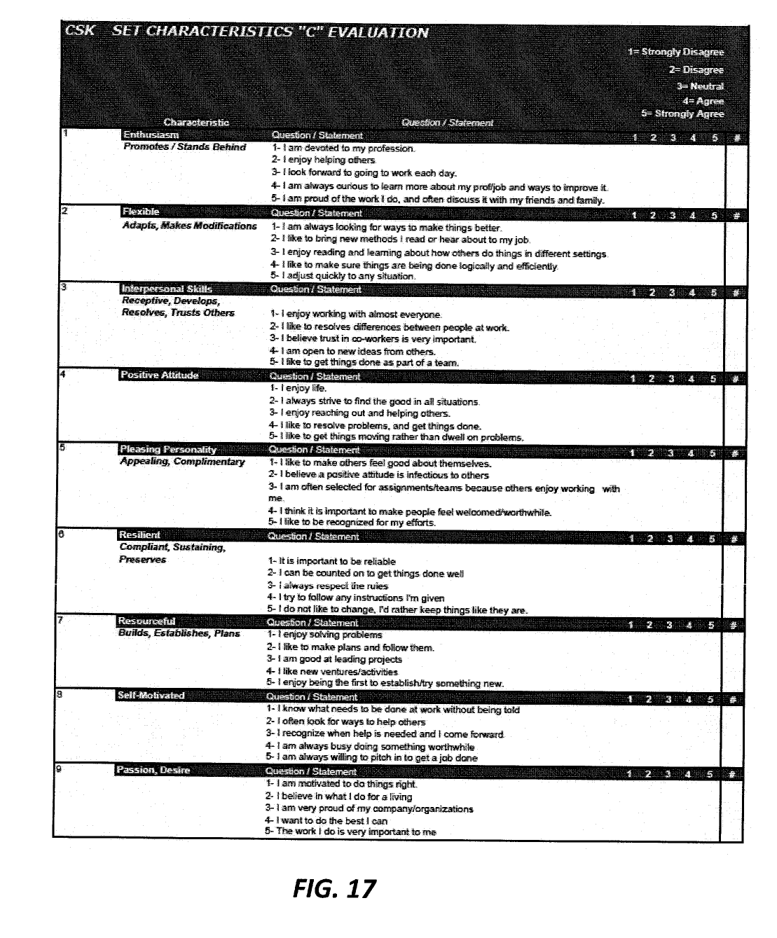

FIG. 7 shows the method for evaluating the characteristics of an individual. The method discussed in FIG. 7 is exemplary only and uses the diagrams shown in FIG. 5(a) and FIG. 5(b) in order to discuss the results. In step 402 an examination for evaluating characteristics is provided to an individual. An example of an examination for evaluating characteristics is shown in FIG. 17. The characteristic examination determines the characteristics of an individual 40(a) such as, enthusiasm, flexible, interpersonal skills, positive attitude, pleasing personality, resilient, resourceful, self-motivated and passion, etc. In step 404 the results of the characteristic test are evaluated. The result of this evaluation usually obtains a number or some form of quantitative result. The quantitative result is indicative of the neural wiring that an individual currently has. In step 406 the results of the evaluation performed in step 404 are compiled with the results of the other individuals 40(b) and 40(c). In step 408 the results are compared with the others. A target number may be used in order to perform the comparison. For example the characteristic test may require a score of 3 on a scale of 1-5 to determine level of extroversion. Two of the individuals may only score a 1 and a 2, while the third may score a 5. This would indicate that the extroversion characteristic of two of the individuals would need to be brought up to parity before formation of the integrated group 50. In step 410 the result is corrected. This may be accomplished by assigning a coach to assist them with improving the area or issue that is determined to be sub-par.

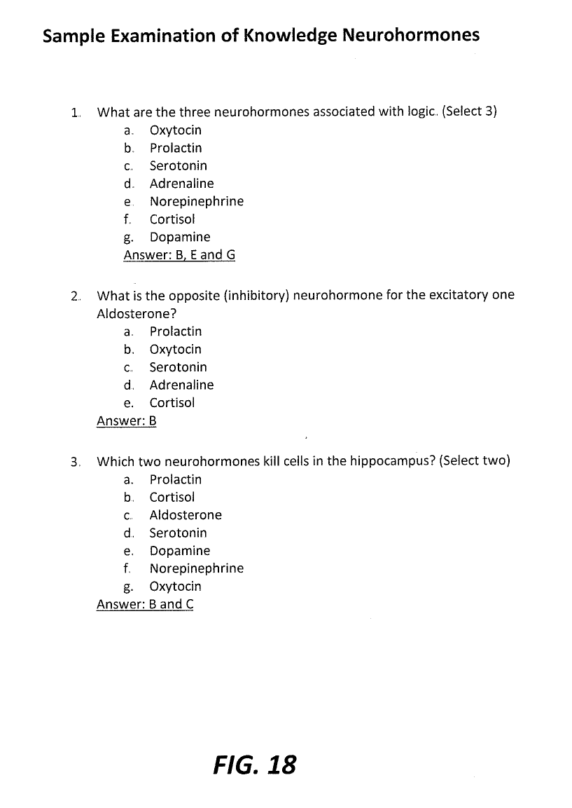

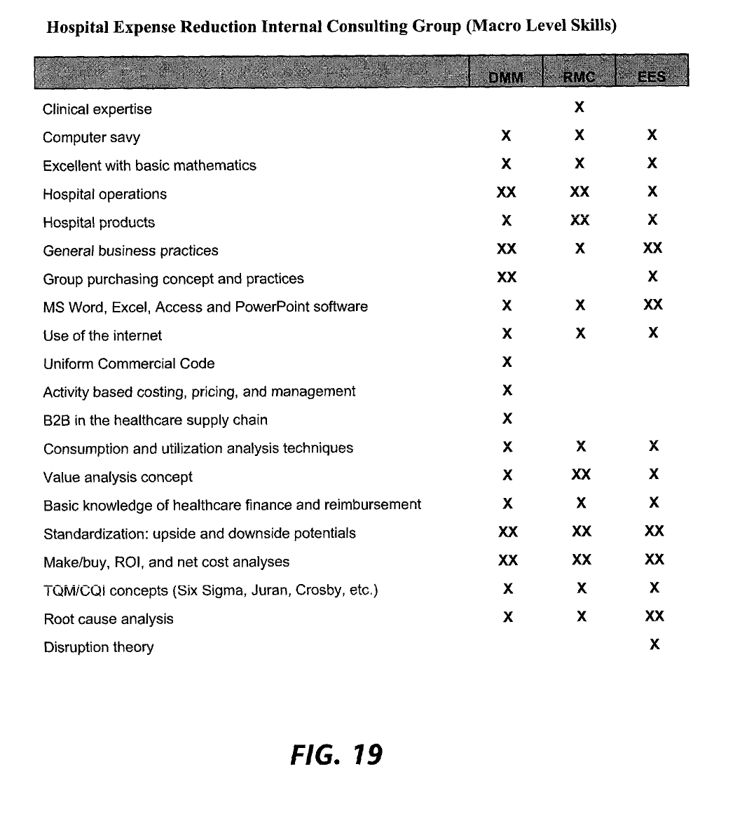

FIG. 8 shows the method for evaluating the knowledge of an individual. The method discussed in FIG. 8 is exemplary only and uses the diagrams shown in FIG. 5(a) and FIG. 5(b) in order to discuss the results. In step 502 an examination for evaluating knowledge is provided to an individual. FIGS. 18 and 19 provide examples of examinations for evaluating knowledge. The knowledge examination determines the knowledge of an individual 40(a) such as, subjects for medical research for example; molecular biology, neuroplasticity, neuroendocrinology, chemistry, medical geology, nutritional sciences, psychology, bacteriology, virology, mycology, oncology, toxicology, neurology, homeopathy, pharmacology, etc. In step 504 the results of the knowledge test are evaluated. The result of this evaluation usually obtains a number or some form of quantitative result. The quantitative result is indicative of the neural wiring that an individual currently has. In step 506 the results of the evaluation performed in step 504 are compiled with the results of the other individuals 40(b) and 40(c). In step 508 the results are compared with the others. A target number may be used in order to perform the comparison. For example the knowledge test may require a score of 75 on a scale of 1-100 to determine the knowledge level of molecular biology. One of the individuals may only score a 60, while the other two may score a 75 and 90. This would indicate that the molecular biology knowledge of one of the individuals would need to be brought up to parity before formation of the integrated group 50. In step 510 the result is corrected. The result may be corrected by an individual assigned to coach and assist them with improving their knowledge about the core issues in molecular biology that would bring them up to par with the remainder of the group.

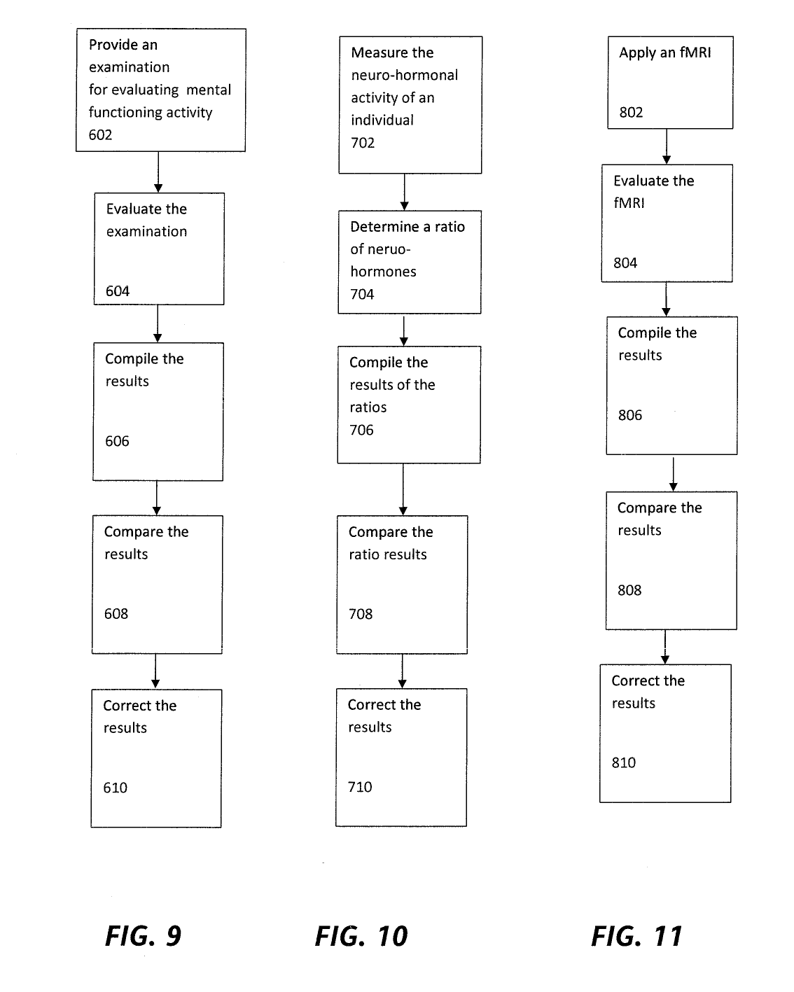

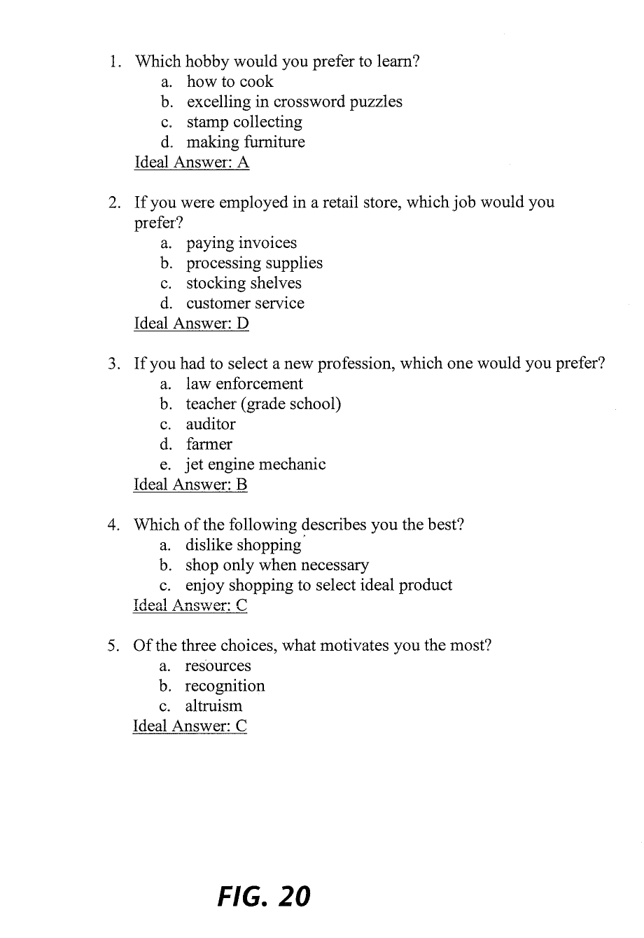

FIG. 9 shows the method for evaluating the MFA of an individual. The method discussed in FIG. 9 is exemplary only and uses the diagrams shown in FIG. 5(a) and FIG. 5(b) in order to discuss the results. In step 602 an examination for evaluating MFA is provided to an individual 40(a), such as that shown in FIG. 4. Another example of this is shown in FIG. 20. The MFA examination determines the MFA an individual such as, focused, impulsive, impulsive and hyperactive, aware, distracted, uninterested and obsessive. In step 604 the results of the MFA test are evaluated. The result of this evaluation usually obtains a number or some form of quantitative result. The quantitative result is indicative of the neural wiring that an individual currently has. In step 606 the results of the evaluation performed in step 604 are compiled with the results of the other individuals 40(b) and 40(c). In step 608 the results are compared with the others. A target number may be used in order to perform the comparison. For example the MFA test may require a score of 11, as scored on the clock paradigm provided above. Two of the individuals may be at 10 and a 2, while the third may score an 11. This would indicate that the MFA of two of the individuals would have to be brought into alignment before formation of the integrated group 50. In step 610 the result is corrected. If an individual's MFA is not in keeping with the desired level, the individual should not be selected to participate in an Internal Consulting Group. Remediation of neural wiring requires considerable time and counseling effort.

FIG. 10 shows the method for evaluating the neuro-hormonal level of an individual. The method discussed in FIG. 10 is exemplary only and uses the diagrams shown in FIG. 5(a) and FIG. 5(b) in order to discuss the results. In step 702 an examination for evaluating the Neuro-hormonal level is provided to an individual. The neuro-hormonal levels are used to obtain ratios of select hormones in an individual 40(a) such as, 1:2, 1:10 etc. In step 704 a ratio of neuro-hormones is obtained. The result of this evaluation usually obtains a number or some form of quantitative result, in this case a ratio. The quantitative result is indicative of the neural wiring that an individual currently has. In step 706 the results of the evaluation performed in step 704 are compiled with the ratio results of the other individuals 40(b) and 40(c). In step 708 the results obtained in step 704 are compared with the others. A predetermined target ratio may be used in order to perform the comparison. For example the ratio may be predetermined to and/or preferred to be 1 part per every 2 parts. Two of the individuals may be at 1 part per every 2 parts, while the third may score 1 part per every 3 parts. This would indicate that the neuro-hormonal level of one of the individuals may be out of balance and would have to be brought into alignment before formation of the integrated group 50. In step 710 the result is corrected. One way of correcting neurohormone levels may be accomplished by the individual themselves based on changing their thought processes. Modification of thought processes may be coached or counseled and the reconfiguration itself may be achieved through reflective meditation or self-awareness. If an individual's thought processes are disrupted by emotional imbalances, these psychological disruptions may be modified by the use of dietary supplements, medications, or the application of specific neuro-hormones intravenously or orally. After the realignment of any existing emotional disruptions, an individual may proceed in the process of reconfiguring their thought processes. After correction, the individual may be retested in order to determine if the correcting procedure is taking effect. Retesting may continue to occur until positive results are obtained or the individual can be disqualified as a candidate for the Internal Consulting Group.

FIG. 11 shows the method for evaluating the fMRIs scans of an individual. The method discussed in FIG. 11 is exemplary only and uses the diagrams shown in FIG. 5(a) and FIG. 5(b), as well as FIGS. 2(a)-3(b) in order to discuss the results. In step 802 an fMRI examination for evaluation is provided to an individual. In step 804 the fMRI scan is evaluated. For example it may look the fMRI scan shown in FIG. 2(a). The result of this evaluation obtains an image scan. This image scan is indicative of the neural wiring that an individual currently has. In step 806 the fMRI image scan obtained in step 804 is compiled with the results of the other individuals. In step 808 the results are compared with the others. A predetermined target image may be used in order to perform the comparison. For example the fMRI scan may be required to be similar to the one shown in FIGS. 2(b) and 3(b). This would indicate that the neural wiring of two of the individuals needs to be brought into particular alignment before formation of the integrated group 50. In step 810 the result is corrected. Reconfiguration of neural wiring may take place when an individual's thoughts are changed based on their habit of thinking. To accomplish task, they must change their thought patterns relative to any issue or topic. This may involve taking more than one fMRI scan and comparing the interim results in order to ascertain whether or not the individual's fMRI scan is getting closer to matching the predetermined fMRI scan.

FIG. 5(b) shows the same individuals 40(a)-40(c) used to form a core group that may be called the integrated group 50. The integrated group 50 may then be further evaluated in order to determine the success of the integration process. This test may involve the same tests discussed above in reference to FIGS. 6-11. The tests may be performed until each of the threshold values are obtained.

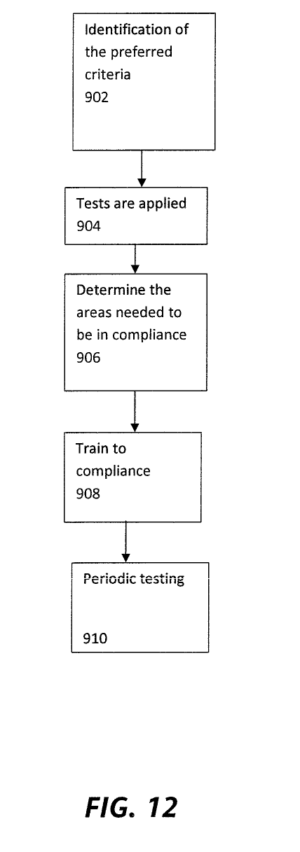

FIG. 12 shows the method used in order to form the integrated group 50. The integrated group 50 is formed via specific selection of common behavior and patterns of thought that are shared by the individuals. In step 902 the preferred criteria for the integrated group 50 are identified. The criteria may involve any one or all of the tests discussed above with respect FIGS. 6-11. In step 904 the specific tests, such as those described above with respect to FIGS. 6-11, are applied. In step 906, the sampling of the individual are then used to determine which specific areas need to be brought into compliance so as to meet the preselected threshold criteria. In step 908, training material and instructional material are then employed to push the individuals 40(a)-40(c) towards forming the integrated group 50 having homogenous behavior and patterns of thought. The homogenous behavior and patterns of thought are created by the participating individuals through the sharing of opinions regarding the guidelines and criteria for the operations of the Internal Consulting Group. The skills acquired by the participants are those shared between them and enhanced through input and consensus regarding their benefits to the objective of the group as a whole. In step 910, ideally the individuals 40(a)-40(c) may be periodically tested in order to determine whether or not they are moving towards the desired goal. While the individual may be trained via structured testing, it may be possible to apply certain neuro-hormones to an individual in order to restore a preferred neuro-hormonal ratio.

The integrated groups 50 can accomplish the goals of establishing a unified culture. An integrated group 50 may adhere to organizational guidelines put forth by a governing body. These guidelines ideally offer minimum specifications and restriction and have a strong focus on the desired objective. The integrated group 50 can accomplish the organization's needed tasks and/or create and oversee a separate “implementation team” to execute the creation of the variety of phrases discussed below. The integrated group 50 can also be used for collectively educating the group through a shared learning process.

The integrated group 50 may then employ shared learning in order to present different perspectives (base knowledge, skills and expertise) from all individuals within the integrated group 50. This provides for the interpretation of various philosophies and theories which might influence the current and future needs of the new culture. The value of the collective education method is to function as the engine to recognize, evaluate and drive the identification and evaluation of viable alternatives.

The integrated group 50 may then employ the education, training and information pertaining to all aspects of a specified task, role, function, or purpose. The integrated group 50 enables members to share, maintain, and grow the acquired skills and knowledge thereby enabling the members to deliver optimal results that pertain to their specified task, role, function, or purpose.

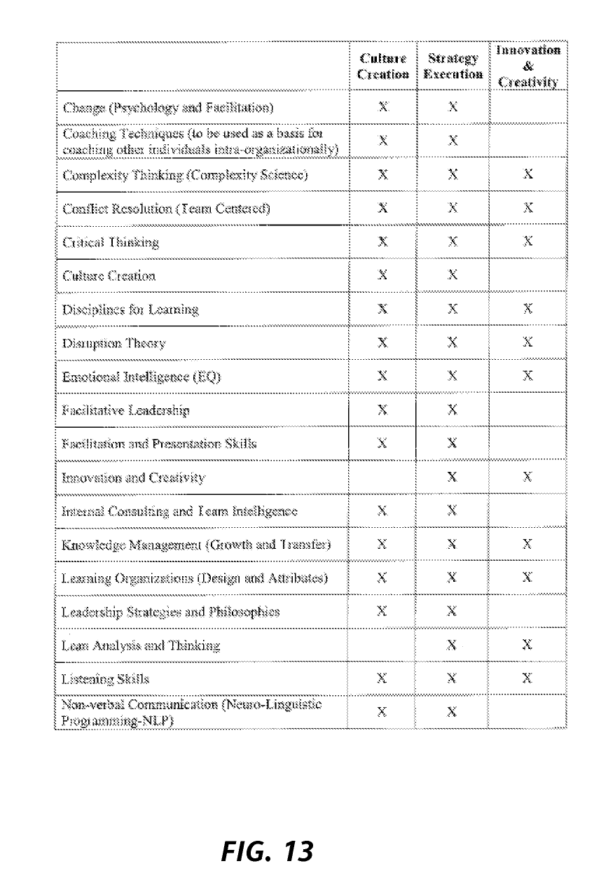

Before integrated group 50 is utilized it is formed in the manner discussed above. FIG. 13 shows an example of some of the information, skills, knowledge, topics and subjects that may be selected for different purposes and that may be pursued by an organization in order to achieve culture creation, strategy execution, innovation and creativity. The items selected for each category may correspond to the needed skills and knowledge as they are related to the purpose. These items can be compared to that of a syllabus used to itemize the various subjects taught in educational settings.

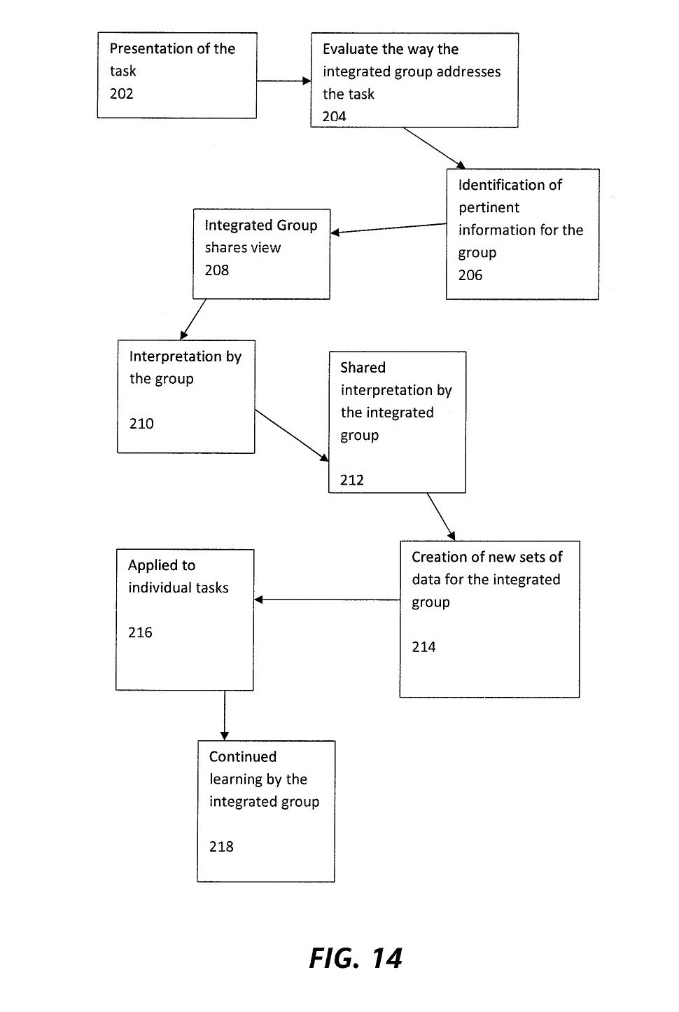

Implementation of the integrated group may involve the steps shown in FIG. 14. The first step in the collective education method assumes that an integrated group 50 has already been formed. The integrated group 50 may be assembled or already exist within an organization.

In general, the specific knowledge, skills and characteristics needed for a given integrated group 50 may be ascertained by the organization. For example, using medical research as an objective of the organization, the following are examples of measurable outcomes: creation of standardized terminology that would eliminate confusion; e.g. gene-protein, cell-receptor on a cell, ligand-substance, etc. The ability of the individuals to be well versed in the targeted disciplines (not necessarily experts in each one). Identifying core roles for individuals; e.g. historian (record keeper); spokesperson. The ability to share opinions across specialties (disciplines) in order to combine knowledge in order to discuss possible linkages between potential disruptions that can lead to a negative outcome. Identifying the point at which an expert must be utilized to explain a specific issue in terms that the entire group can understand. Cultivating the skills of the participating individuals to correct breakdowns in communication as they occur. The integrated group 50 may then be tasked with solving a problem or accomplishing a specific task. Smaller integrated groups 50 of two or more may utilize the collective education method for taking on less complicated task, or objectives. Large integrated groups can use the collective education method for such complex purposes as culture creation, strategy execution, likely evolution, and creativity and innovation.

An example of what an integrated group 50 can be used for is to develop a shared vision for an organization. Typically when companies merge, participants in the planning process often struggle to establish a strategic vision, which provide an overarching statement summarizing the ideal direction the parties should take. Historic differences and the ideology of the individual leaders of the respective parties often prevent effective collaboration between or within organizations.

The seed for collaboration must come from interfacing all necessary cultures while protecting their individual core ideologies without demeaning the core ideologies of the other cultures. While this critical phase of the culture creation process may be difficult, it can be accomplished by the collaborative efforts of a multi-cultural cross-functional team such as the integrated group 50.

Another use of the integrated group 50 is for creating corporate change. Rarely do organizations recognize that their failed efforts to build intra-organizational collaboration care attributable to cultural differences. Interdepartmental or corporate politics and the creation of “silos” are, in fact, clashes between different cultures within the organization. Accordingly, until such cultural differences are addressed, the chances for meaningful, long term harmony and intra-organizational collaboration are minimal.

Another use the integrated group 50 is for providing a shared fate. The shared fate transcends the mere preparation of a statement for culture-wide or cross-cultural dissemination. Rarely have organizations provided a core group of their mid-level leaders/managers with the authority and support to create processes to execute strategic changes required for establishing a new culture. Unlike historic approaches for attempting to modify current attitudes, opinions and differences, the process of culture creation necessitate the delineation of all deficiencies, obstacles and weakness which impact an organization's ability to implement change. By working together with the guidance of unbiased third part facilitator(s), the implementation team can create internally prepared action plans to create new mechanisms or processes which address all of the issues to the satisfaction of all organizations. This planning/problem identification process is the creation of a value proposition. The task of identifying what points or factors offer each organization the features or outcomes that deliver all parties the necessary “wins”. The outcome of this exercise is the identification of WIIFMs (What's In It For Me) that each organization recognizes as being powerful enough to get their members to be willing to accept behavioral change/modification—while acknowledging and respecting the need for the other (cultures) to obtain their WIIFMs without sacrificing their own core ideologies.

FIG. 14 illustrates the steps used in shared learning by the integrated group 50. At step 202, an initial task is provided to the integrated group 50. The integrated group 50 may then address the issues presented by the task.

The presentation of the task in step 202 should include a broad overview of the subject matter, the various topics which will be addressed in forthcoming sessions, and an explanation of how this material will apply specifically to the objectives of integrated group 50 individually and as a whole to meet their objectives.

After the initial screening, selection and formation of the integrated group 50, the integrated group 50 is provided with educational and training material. An estimated timeline for the completion of all individual assignments as well as the completion of and the education and training associated with the subject matter. However, as part or step 202, the integrated group may be provided with several articles or other background documents which clearly indicate the benefits and potential of applying the newly acquired skills and knowledge to the objectives of their task, role, or occupation. Such documents could be assigned as homework and used to initiate discussions at the next session.

At step 204, the individuals 40(a)-40(c) of integrated group 50 will have individually evaluated and ascertained the way to address the initial task. Each individual of the integrated group 50 will have been reviewed the material pertinent to the task provided in step 202. For example, the subject of team building would be sub-divided into individual roles, establishing behavioral norms, conflict resolution, brainstorming techniques, meeting management, etc. As part of step 204, integrated group 50 should attempt to interpret information provided as a starting point for the establishment of a foundation of their skills or knowledge as it can apply to their particular task, role, or occupation.

At step 206, the integrated group 50 identifies all pieces of new information within each topic which may be useful in having them perform their task, role, or occupation more effectively or efficiently. Discussions within the integrated group 50 may be held in order to share the new information to gain insight into the applicability of the information as it is perceived by others as being applicable to their needs and objectives. The purpose of these topic-by-topic information gathering sessions for each subject is for the integrated group 50 to gain a broad understanding of how individual issues and information can be viewed as being valuable pieces for potential use in order to create comprehensive solutions as opposed to mere incremental changes of enhancements based on the views or opinions of only a select few individuals.

At step 208, integrated group 50 shares the view of the individuals 40(a)-40(c). As part of this phase, the integrated group 50 attempts to interpret and share the knowledge and insight gained through the collaborative sharing of respective views, perspectives and interpretations of the other participants as it pertains to their respective task, role, or occupation. This joint process is intended to identify previously unrecognized possibilities or opportunities by combining various elements of information and converting it into intelligence which pertains to the subject matter (i.e., in this sense, intelligence refers to the type of collective acumen that can be built by combining various elements of information).

At step 210 interpretation by the integrated group 50 is performed. At this step the members 40(a)-40(c) separate in order to independently review and reflect upon all of the newly acquired and created intelligence. This independent review by the members 40(a)-40(c) is designed to allow the members 40(a)-40(c) adequate time to analyze how the newly created intelligence may affect their task, role, or occupation as well as its possible impact on the object of the collective education method and system. At this step, each subject is addressed as part of the collective education concept. Accordingly, depending upon that objective, the members 40(a)-40(c) may be required to repeat the aforementioned process for numerous subjects in order to establish a foundation of skills and knowledge from which they can build optimal solutions for assigned tasks or objectives.

At step 212, a shared interpretation is provided by the integrated group 50. The collective education method may be used to build a foundation of skills and knowledge (as opposed to addressing a single subject), the integrated group should reconvene after completing interpretation of the intelligence phase on a subject-by-subject basis. The integrated group 50 can organize the collective education method in such a way so as to insure the subjects are studied in a linear fashion (i.e., the subject acts as a prerequisite for the next). For example, prior to addressing time management, the integrated group 50 should receive education and training in listening skills as well as topics pertaining to when and how to learn, study, and interpret information optimally. The integrated group 50 integrates all of the knowledge accumulated (i.e. from interfacing the knowledge gained from combining the intelligence from the first and second subject, from the first through the third subject, etc.). The integrated group 50 may revisit the accumulated base of expertise and update it as it pertains to individual tasks, roles, or occupations as well as its applicability to the objective of the collective education method as a whole following the completion of the education and training of each of the designated subjects.

Upon reconvening, the integrated group 50 may document specific intelligence gained as it pertains to possible alternatives as to how material within the subject is handled and how the learning itself is handled. This process of using collaboration to build upon or enhance state-of-the-art practices or theories on a subject-by-subject basis to the point when they can be interfaced to contribute to the creation of the next iteration of a process, task, or objective is critical to ensure organizations build, maintain, and grow intellectual capital in order to ensure the optimal execution of strategies.

Step 214, involves the creation of “new” skill and knowledge sets for the integrated group 50 and, when applicable to the integrated group 50 as a whole. Following the use of the collective education method with each subject, the individuals 40(a)-40(c) are prepared to address their assigned objectives by using their new collective skill and knowledge sets. They will have gained the collective acumen to create solution or options capable of meeting the tasks assigned to the integrated group 50.

In step 216, the learned results from the operation of the integrated group are applied to individual tasks. Ideally, the task is accomplished.

In step 218, the results obtained through the collective education method are capitalized upon in order to continue the overall learning process, while maintaining the continued development of the learning process. While the integrated group 50 will gain the knowledge to create optimal solutions through empowerment by the leadership, they must remain committed to the constant maintenance of the new process by revisiting the entire process on an ongoing or predetermined schedule. This knowledge growth process can be assigned to individuals within the integrated group 50 per subject to research. The subject and all associated sub-topics and issues should be researched on an ongoing basis in order to determine when additions or enhancements to the then current state-of-the-art knowledge (based on the outcome of the collective education method and system) should be updated. While any member of the integrated group 50 may identify new information which should be injected into the process, any such new infoujiation should be directed those who are responsible for that category of information. Then, depending upon the protocols established at the conclusion of the collective education method, the integrated group 50 should circulate or present this new information to the other members on an ongoing basis or a predetermined schedule.

It is to be understood, however, that even though numerous characteristics and advantages of the present invention have been set forth in the foregoing description, together with details of the function of the invention, the disclosure is illustrative only, and changes may be made in detail within the principles of the invention to the full extent indicated by the broad general meaning of the terms in which the appended claims are expressed.

What is claimed is:

1. A method for testing for an integrated group comprising the steps of:
measuring a first neuro-hormonal level of an first individual;
determining a first ratio of neuro-hormones based upon the first neuro-hormonal level;
compiling a plurality of ratios, wherein the first ratio of neuro-hormones is one of the plurality of ratios;
comparing the first ratio with a predetermined ratio; and
correcting the first ratio of the first individual.

2. The method of claim 1, wherein the step of correcting is selected from the group consisting of coaching, counseling the individual, and applying a medication, herbal supplement, nutritional product or administering a neuro-hormone.

3. The method of claim 2, further comprising re-measuring the neuro-hormonal level of the first individual.

4. The method of claim 3, wherein the step of correcting further comprises matching the ratio of the first individual to the predetermined ratio.

5. The method of claim 1, further comprising measuring a plurality of neuro-hormonal levels.

6. The method of claim 5, further comprising measuring a second neuro-hormonal level of a second individual.

7. The method of claim 6, further comprising determining a second ratio of neuro-hormones based upon the second neuro-hormonal level.

8. The method of claim 7, further comprising comparing the second ratio with the pre-determined ratio.

9. The method of claim 8, further comprising correcting the second ratio, wherein correction of the second ratio requires matching the second ratio of the second individual to the predetermined ratio.

10. The method of claim 1, further comprising applying an fMRI reading to a first individual in order to obtain a scan.

11. A method for testing for an integrated group comprising the steps of:
applying an fMRI reading to a first individual in order to obtain a first scan;
evaluating the first fMRI scan;
compiling the results of a plurality of fMRI scans;
comparing the first fMRI scan with a predetermined fMRI scan; and
correcting the first individual with respect to the predetermined fMRI scan.

12. The method of claim 11, wherein the step of correcting is selected from the group consisting of coaching, counseling and applying a medication, herbal supplement, nutritional product or administering a neuro-hormone.

13. The method of claim 12, further comprising obtaining a second fMRI scan of the first individual.

14. The method of claim 13, wherein the step of correcting further comprises matching the second fMRI scan to the first fMRI scan.

15. The method of claim 14, wherein the step of correcting further comprises determining if the second fMRI scan is closer to matching the predetermined fMRI scan.

16. The method of claim 11, further comprising obtaining a third fMRI scan of a second individual.

17. The method of claim 16, further comprising comparing the third fMRI scan to the first fMRI scan.

18. The method of claim 17, further comprising comparing the third fMRI with the predetermined fMRI.

19. The method of claim 18, further comprising correcting the second individual.

20. The method of claim 11 measuring a first neuro-hormonal level of a first individual.
